(19) United States
(12) Patent Application Publication
(10) Pub. No.: US 2021/0053903 A1
(43) Pub. Date: Feb. 25, 2021

(54) APPLYING HANSEN SOLUBILITY PARAMETERS TO METAL-ORGANIC FRAMEWORK SYNTHESIS CONDITIONS

(71) Applicant: ExxonMobil Research and Engineering Company, Annandale, NJ (US)

(72) Inventors: Carter W. Abney, Califon, NJ (US); Anna C. Ivashko, Denville, NJ (US)

(21) Appl. No.: 16/900,109

(22) Filed: Jun. 12, 2020

Related U.S. Application Data
(60) Provisional application No. 62/890,645, filed on Aug. 23, 2019.

(51) Int. Cl. C07C 65/105; C07F 15/06

(52) U.S. Cl. CPC C07C 65/105; C07F 15/06

(57) ABSTRACT

A method suitable for use in investigating synthesis conditions for metal-organic frameworks may include: identifying synthesis conditions that includes a first solvent system by which a metal-organic framework is successfully synthesized; and synthesizing the metal-organic framework under substantially the same synthesis conditions but with a second solvent system (or a comparable solvent system) having a difference (Rₐ) between Hansen solubility parameters of 5 MPa^{0.5} or less as compared to the first solvent system.

### BACKGROUND

The present disclosure relates to synthesizing metal-organic frameworks (MOFs).

Metal organic frameworks (MOFs) are crystalline compounds consisting of metal ion/oxide secondary building units (SBUs) interconnected by rigid organic molecules to form one-, two-, or three-dimensional structures that can be porous. MOFs are characterized by low densities, high internal surface areas, and uniformly sized pores and channels. For example, U.S. Pat. No. 8,653,292 describes Zr MOFs having a surface area of at least 1020 m²/g or, if functionalized, having a surface area of at least 500 m²/g. As a result of these advantageous properties, MOFs have been investigated extensively for applications in gas separation and storage, sensing, catalysis, drug delivery, and waste remediation. For example, in some cases, guest molecules can stably enter the pores, thus MOF crystals can be used for the storage of gases (e.g., H₂ and CO₂). Further, since some guest molecules can enter more easily than others, and the pores can be functionalized to change their chemical properties, this can be used as the basis for separation methodologies. For example, MOFs can be used to make highly selective and permeable membranes to separate small gas molecules (e.g., CO₂ from CH₄) or liquid molecules (e.g., hydrocarbons, alcohols, water). Additional applications of MOFs are in catalysis, in drug delivery, and as sensors.

The wide array of potential applications for MOFs stem from the nearly infinite combination of organic linkers and SBUs available. Because of the multiple conformations possible between the linker and metal SBUs, predicting and directing structure is challenging. Synthetic screening protocols typically employed for MOF research use a brute force approach and test multiple synthesis conditions (e.g., time, reactant concentrations, and solvent composition) in parallel. Once a desired MOF product is identified, the associated synthesis conditions are rarely altered. This is especially true for the solvents because it has been observed that the solvent composition can have a significant effect on whether a MOF can be synthesized and the morphology of the MOF synthesized. Therefore, if a desirable MOF is originally synthesized with an expensive and/or toxic solvent, the scaled-up manufacturing of the MOF is performed with said solvent at a higher cost of manufacturing and/or additional safety protocols.

### SUMMARY OF THE INVENTION

The present disclosure relates to synthesizing MOFs and using Hansen solubility parameters of the solvent to identify a workable range for the Hansen solubility parameters, identify alternative solvents, and tune MOF morphology.

A nonlimiting aspect of the present disclosure is a method comprising: identifying synthesis conditions that includes a first solvent system by which a metal-organic framework is successfully synthesized; and synthesizing the metal-organic framework under substantially the same synthesis conditions but with a second solvent system (or a comparable solvent system) having a difference (Rₐ) between Hansen solubility parameters of 5 MPa^{0.5} or less as compared to the first solvent system.

Another nonlimiting aspect of the present disclosure is a method comprising: performing a plurality of metal-organic framework syntheses under two or more of substantially the same synthesis conditions that vary a solvent composition based on a Hansen solubility parameter; and identifying a range for the Hansen solubility parameter under which the metal-organic framework can be synthesized.

Yet another nonlimiting aspect of the present disclosure is a method comprising: performing a plurality of metal-organic framework syntheses under two or more of substantially the same synthesis conditions that vary a solvent composition based on a Hansen solubility parameter; characterizing a morphology characteristic of the metal-organic frameworks; identifying trends in the morphology characteristic as a function of the Hansen solubility parameter; and synthesizing a metal-organic framework under substantially the same synthesis conditions with a solvent having the Hansen solubility parameter chosen to achieve a desired morphology characteristic based on the identified trends.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIGS. 1-3 are plots of the three Hansen solubility parameters for select polar solvent compounds, select nonpolar solvent compounds, and mixed solvents, respectively.

FIG. 4 includes scanning electron micrographs of the MOF-274 synthesized under the same synthesis conditions except with different solvents having comparable Hansen solubility parameters.

FIG. 5 is a plot of the powder x-ray diffraction data for the MOF-274 synthesized under the same synthesis conditions except with different solvents having comparable Hansen solubility parameters.

FIG. 6 is a plot of the surface area for the MOF-274 synthesized under the same synthesis conditions except with different solvents having comparable Hansen solubility parameters.

FIG. 7 illustrates the Hansen solubility parameters for a series of solvents with a comparable δ_{D}, a comparable δ_{P}, and a greatly varying δ_{H}.

FIG. 8 illustrates the Hansen solubility parameters for a series of solvents with a comparable δ_{H}, a comparable δ_{H}, and a greatly varying δ_{P}.

FIG. 9 illustrates the boundary Hansen solubility parameters defined by FIGS. 7 and 8.

FIGS. 10 and 11 are scanning electron micrographs and a plot of the powder x-ray diffraction data, respectively, for MOF-274 synthesized with variable δ_{H}.

FIGS. 12 and 13 are scanning electron micrographs and a plot of the powder x-ray diffraction data, respectively, for MOF-274 synthesized with variable δ_{P}.

FIG. 14 includes scanning electron micrographs of MOF-274 synthesized in 10% water, 25% toluene, and 65% DMSO.

### DETAILED DESCRIPTION

The present disclosure relates to synthesizing MOFs and using Hansen solubility parameters of the solvent to identify a workable range for the Hansen solubility parameters, identify alternative solvents, and tune MOF morphology.

Hansen solubility parameters are developed from the Hildebrand solubility parameter, which relates solubility to the square root of cohesive energy density. A shortcoming of defining solubility as a single parameter, as presented by Hildebrand, is that is fails to account for association between molecules, such as arising from polarity or hydrogen-bonding interactions. In contrast, Hansen solubility parameters are predicated upon the understanding that the total energy of vaporization, and thus the total cohesive energy, consists of several individual parts, arising from atomic dispersion forces, dipole-dipole forces between permanent dipoles, and hydrogen bonding. Accordingly, there are three Hansen solubility parameters, each typically measured in MPa^{0.5}: the energy from dispersion forces between molecules (also referred to as a dispersion parameter or δ_{H}), the energy from a dipolar intramolecular force between molecules (also referred to as a polarity parameter or δ_{P}), and the energy from hydrogen bonds between molecules (also referred to as a hydrogen bonding parameter or δ_{H}). Materials possessing similar Hansen solubility parameters have high affinity for each other, with the extent of similarity determining the extent of interaction. Thus, Hansen solubility parameters provides a more quantifiable means to understand the adage “like dissolves like,” and is often used to predict if a first material will dissolve in a second material to form a solution. See Hansen Solubility Parameters: A User's Handbook. Charles M. Hansen. CRC Press, Boca Raton, Fla. 2007. 2^{nd} Ed.

Hansen solubility parameters for common solvent compounds (e.g., acetone, methanol, dimethyl sulfoxide (DMSO), toluene, cyclohexane, and the like) are well known and readily available, for example in the commercially available HSPiP database. Hansen solubility parameters can be determined experimentally for a new molecule by dispersing the molecule in a series of solvents with known Hansen solubility parameters. Upon identifying which solvents dissolve the molecule, the Hansen solubility parameters for these solvents are plotted using Cartesian coordinates by assigning each parameter to its own axis, affording a sphere of solubility in what is referred to as “Hansen space.” The center of the sphere of solubility defines the empirical Hansen solubility parameters for the molecule. Further, the Hansen solubility parameters for a solvent mixture can be calculated by volume-weighted averaging of the Hansen solubility parameters for each component in the mixture.

As used herein, the term “solvent” refers to the liquid in which a solute is dissolved and/or dispersed and encompasses mixtures of liquids unless otherwise specified.

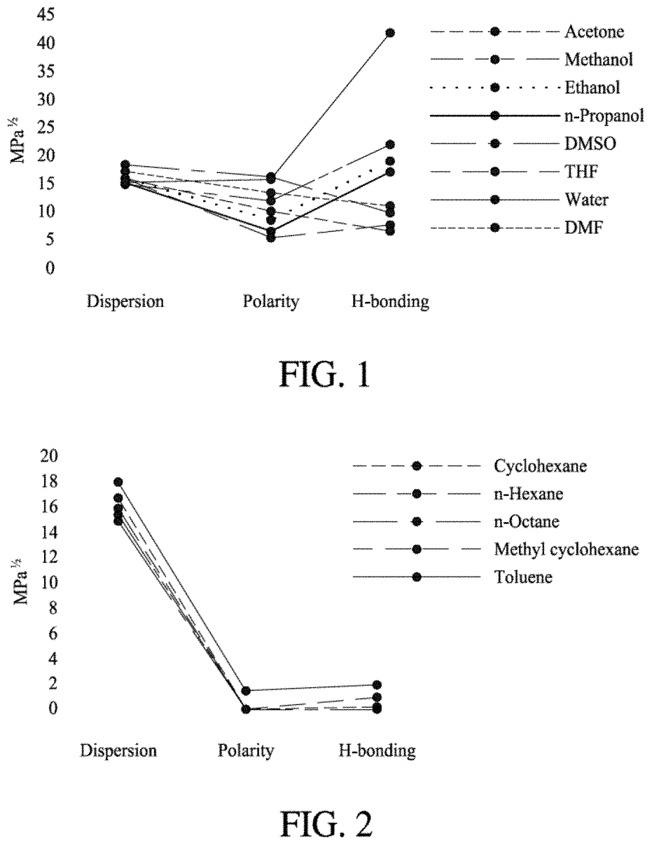

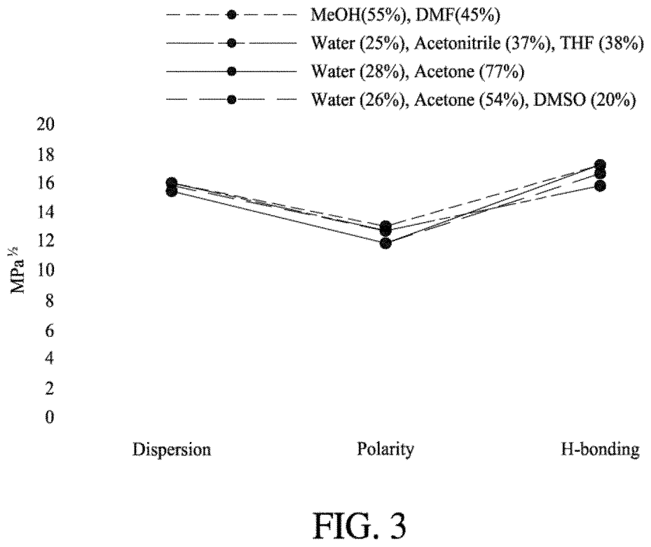

FIGS. 1-3 are plots of the three Hansen solubility parameters for select polar solvent compounds, select nonpolar solvent compounds, and mixed solvents, respectively.

The present disclosure approaches screening of the MOF synthesis conditions, specifically solvent composition, from a perspective of varying Hansen solubility parameters rather than a combinatorial compositional approach. That is, in typical brute force approaches, a variety of solvents are used based on availability and researcher experience. In contrast, the present disclosure equates solvents with comparable Hansen solubility parameters, which reduces the number of solvents that need to be tested. Further, such an approach allows for changing solvents for given MOF synthesis conditions, where the alternative solvent advantageously may have a lower cost and/or a lower toxicity. Other advantages and applications of the Hansen solubility parameters in MOF synthesis are described herein.

Prospective solvent compositions are identified by minimizing the difference in δ_{D}, δ_{P}, and δ_{H} between the original solvent or solvent system, S₁, and the new multi-component solvent system, S₂. As defined in Hansen Solubility Parameters: A User's Handbook, Charles M. Hansen, CRC Press, Boca Raton, Fla. 2007. 2^{nd} Ed., the difference, Rₐ, is traditionally defined by the following equation.

(Rₐ)²=4(δ_{D2}−β_{D1})²+(δ_{P2}−δ_{P1})²+(δ_{H2}−δ_{H1})²

where δ_{D1} and δ_{D2} are the dispersion parameters for the first and second solvents, respectively; δ_{P1} and δ_{P2} are the polarity parameters for the first and second solvents, respectively; and δ_{H1} and δ_{H2} are the hydrogen bonding parameters for the first and second solvents, respectively.

More solvent systems that are mixtures of two or more liquids, the values of δ_{D2}, δ_{P2}, and δ_{H2} are determined by taking the sum of the constituent solvents weighted by their percent volume comprising S₂. In other words a representative Hansen solubility parameter for a solvent system S₂ comprising n different solvents is defined by the following equation.

δ=v₁×δ₁+v₂×δ₂+v₃×δ₃+ . . . +vₙ₋₁×δₙ₋₁+vₙ×δₙ

where vᵢ refers to the percent volume of solvent i in S₂, and δᵢ refers to the corresponding Hansen solubility parameter for solvent i.

A minimum value for Rₐ between S₁ and any S₂ can thus be obtained by varying the volume of solvents comprising S₂. The number of solvents included in S₂, n, is not limited. However, the sum of the percent volume of solvents, v₁+v₂+v₃+ . . . +vₙ₋₁+vₙ must equal 100%, and refers to the sum of the volume of the individual solvents rather than the volume of the solvents after mixing.

As used herein, the term “comparable Hansen solubility parameter,” whether general or specific to a single Hansen solubility parameter, refers to the Hansen solubility parameter being within 10%. For example, when describing two Hansen solubility parameters (δ₁ and δ₂) being comparable, the Hansen solubility parameters fulfill

$$10 \% \leq \frac{( {δ}_{1} - {δ}_{2} )}{{δ}_{1}} ,$$

where δ₁≥δ₂ and the two Hansen solubility parameters are the same parameters (i.e., δ₁ and δ₂ both being a δ_{H} or a δ_{P} or a δ_{H}). Where δ₁=δ₂, the Hansen solubility parameters are the same and therefore, considered comparable. Preferably, comparable Hansen solubility parameters are with 5% of each other. The Hansen solubility parameters and Rₐ derived therefrom are measured and/or calculated typically at room temperature, but at reaction temperature or any other suitable temperature may be used. When comparing Hansen solubility parameters and Rₐ derived therefrom, the comparison should be for Hansen solubility parameters and Rₐ at the same temperature.

As used herein, a “comparable solvent system” refers to when the Rₐ (difference in Hansen solubility parameters for the solvent system) calculated for any two solvent systems S₁ and S₂ of 5 MPa^{0.5} or less (or 2 MPa^{0.5} or less, or 1 MPa^{0.5} or less or 0 MPa^{0.5} to 5 MPa^{0.5}, or 0 MPa^{0.5} to 2 MPa^{0.5}, or 0 MPa^{0.5} to 1 MPa^{0.5}).

Molecular volume comprises a fourth parameter that can be considered in the design of alternative solvents for MOF synthesis or establishment of a range of synthetic conditions, with solvents with smaller molecular volume typically regarded as superior to those with larger molecular size.

Examples of solvents that can be used alone or in solvent mixtures include, but are not limited to, acetone, acetonitrile, benzyl alcohol, 1-butanol, 2-butanol, n-butyl acetate, cyclohexane, cyclohexanol, cyclohexanone, diacetone alcohol, 1,4-dioxane, methanol, ethanol, ethyl acetate, ethyl benzene, ethyl lactate, ethylene carbonate, ethylene glycol, ethylene glycol monobutyl ether, ethylene glycol monomethyl ether, gamma-butyrolactone (GBL), heptane, hexane, n-propanol, iso-propanol, n-butanol, iso-butanol, t-butanol, iso-propyl acetate, isophorone, d-limonene, methyl acetate, methyl ethyl ketone, N-methyl-2-pyrrolidone (NMP), methylene chloride, 1-nitropropane, n-propyl acetate, propylene carbonate, water, dimethyl sulfoxide (DMSO), tetrahydrofuran (THF), water, N,N-dimethylformamide (DMF), N,N-dimethylacetamide, 1,3-dimethylpropyleneurea, hexamethylphosphoramide, dimethyl ether, diethyl ether, methylethylether, pentane, benzene, cyclohexane, n-hexane, n-octane, kerosene, dodecane, methyl cyclohexane, toluene, and the like.

Depending on the solvent, the δ_{D} may range from 5 MPa^{0.5} to 20 MPa^{0.5}, the δ_{P} may range from 0 MPa^{0.5} to 20 MPa^{0.5}, and the δ_{H} may range from 0 MPa^{0.5} to 50 MPa^{0.5}.

The Hansen solubility parameters can be used in several ways to assist with MOF synthesis.

In the first two examples, MOFs are synthesized under two or more synthesis conditions varying solvents and maintaining other synthesis conditions (e.g., reactant concentrations, nominal pH, temperature, and time) substantially the same (i.e., at values that do not materially affect the structure of the MOF synthesized, for example, do not affect the value of a morphology characteristic by more than 10% or by more than the standard deviation of said characteristic, whichever is larger). Herein, said conditions are simply referred to as two or more synthesis conditions varying solvents (or solvent composition) or a grammatical variation thereof.

In a first example, Hansen solubility parameters can be used for identifying a range for the Hansen solubility parameters under which a MOF can be synthesized. In this example, MOFs can be synthesized under two or more synthesis conditions varying solvents such that two of the three Hansen solubility parameters are comparable and the third Hansen solubility parameter is systematically varied (e.g., solvents having a comparable δ_{D}, a comparable δ_{P}, and a varied δ_{H}) to identify a range of the third Hansen solubility parameter under which the MOF can be synthesized. The range of one, two, or all three Hansen solubility parameters can be investigated per the foregoing stipulation so that a synthetic range for the desired Hansen solubility parameters can be determined. In some instances, the determined synthetic range for one or more of the Hansen solubility parameters may be limited by the solvent choices of the researcher. That is, the full synthetic range may extend beyond the experimental range of the Hansen solubility parameters. This could be due to a desire to investigate a smaller experimental range, the availability of solvents, infrastructure limitations, safety considerations, and other factors.

Once a range for the Hansen solubility parameter under which the metal-organic framework can be synthesized has been identified, the MOF may be synthesized with different solvents having the specified Hansen solubility parameter within the corresponding range identified.

When determining a range for the Hansen solubility parameter under which the metal-organic framework can be synthesized, the composition of the solvents can vary to achieve the desired Hansen solubility parameter. For example, while maintaining a comparable δ_{D} and a comparable δ_{H}, the values for δ_{P} can be systematically tuned by varying the volume percentages of solvents used for the synthesis. Solvents may include water, acetonitrile, and THF mixtures at varying relative volume percentages; water, acetonitrile, THF, acetone, and toluene mixtures in varying volume percentages; water, toluene, n-propanol, and ethanol in varying volume percentages; and water, toluene, and n-propanol.

In another application, the Hansen solubility parameters can be used to control or otherwise tailor the morphology of an MOF. For example, as described in the first application above, a plurality of solvents can be tested within the range for the Hansen solubility parameters under which a MOF can be synthesized. The morphology of the synthesized MOFs can be characterized by techniques such as aspect ratio (e.g., via scanning electron microscopy), surface area (e.g., via nitrogen gas adsorption), pore size (e.g., via nitrogen gas adsorption), crystal structure (e.g., via powder x-ray diffraction), composition (e.g., via nuclear magnetic resonance (NMR) spectroscopy (e.g., ¹³C NMR and/or ¹H NMR)), and the like. The trends in morphology changes relative to a change in each of the Hansen solubility parameters may be characterized and used to identify preferred Hansen solubility parameters that correspond to a desired morphology. Using the Hansen solubility parameters in this way allows researchers to test a fewer number of samples, identify morphology trends relative to Hansen solubility parameters, and control or otherwise tailor the morphology based on the identified trends. This drastically contrasts the combinatorial approach currently employed were a variety of solvents are used in screening synthesis conditions with no direction and how to navigate the solvent space.

Yet another application of Hansen solubility parameters in MOF synthesis is the ability to change the solvent to a comparable solvent system with a reasonable expectation of minimal effect on the MOF properties. This can be achieved by minimizing the value for Rₐ as described above.

Therefore, at any point in the process of screening, scale-up, and existing manufacturing, a comparable solvent system can be substituted for the existing solvent where the comparable solvent system affords an Rₐ of 5 MPa^{0.5} or less (or 2 MPa^{0.5} or less, or 1 MPa^{0.5} or less or 0 MPa^{0.5} to 5 MPa^{0.5}, or 0 MPa^{0.5} to 2 MPa^{0.5}, or 0 MPa^{0.5} to 1 MPa^{0.5}). This approach allows for changing solvents for given MOF synthesis conditions, where the alternative solvent advantageously may have a lower cost, a lower toxicity, or otherwise enable greater process intensification for the MOF synthesis, isolation, purification, characterization enhancement, or performance improvement.

While the Hansen solubility parameters of the alternative solvent may be within 10% of the existing solvent Hansen solubility parameters, preferably corresponding parameters between the alternative solvent and the existing solvent are within 5% of each other.

The MOFs described herein are synthesized under known synthesis conditions like time, temperature, reactant concentrations, and the like. Nonlimiting examples of MOF syntheses can be found in U.S. Pat. No. 8,653,292 and US Patent Appl. Pub. Nos. 2007/0202038, 2010/0307336, and 2016/0031920, each of which are incorporated herein by reference. Other nonlimiting examples of MOF syntheses can be found in J. Am. Chem. Soc. 2012, 134, 7056-7065; Nature, 2015, 519, 303-308; Chem. Sci, 2018, 9, 160-174.

By way of nonlimiting example, MOFs can be synthesized by dissolving one or more metal salts with one or more organic linkers at a molar ratio of total metal salts to total organic linkers of 0.2:1 to 5:1 (or 0.6:1 to 3:1, or 0.8:1 to 2:1, or 1:1) in a solvent to produce a reaction mixture. Optionally, an acid or base may be included in the reaction mixture to facilitate the synthesis.

Optionally, an acid and its conjugate base, or a base and its conjugate acid, may be included in the reaction mixture to buffer the nominal pH. These buffers may also be generated in situ by addition of the buffering acid followed by addition of a basic solution to the appropriate pH. Similarly, these buffers may be generated in situ by addition of the buffering base followed by addition of an acidic solution to the appropriate pH. Similarly, these bases can be added as the counterion for the metal source. These acids can be added as the counterion for the deprotonated linker.

As described previously, the acid, base, or buffer are not considered part of the solvent system described herein.

The reaction mixture is generally heated to 50° C. to 175° C. (or 100° C. to 160° C., or 115° C. to 145° C.) for 1 hour to 7 days (or 6 hours to 5 days, or 12 hours to 3 days, or 1 minute to 30 minutes). The reaction mixture is typically then centrifuged to collect the MOFs and washed.

Examples of metals suitable for use in the metal salts include, but are not limited to, lanthanum, cerium, praseodymium, promethium, samarium, europium, gadolinium, terbium, dysprosium, holmium, erbium, thulium, ytterbium, lutetium, aluminum, gallium, indium, magnesium, calcium, strontium, barium, iron, niobium, scandium, yttrium, zirconium, titanium, vanadium, chromium, manganese, cobalt, nickel, copper, zinc, molybdenum, technetium, ruthenium, rhodium, palladium, silver, cadmium, lutetium, hafnium, tantalum, tungsten, rhenium, osmium, iridium, platinum, gold, mercury, lawrencium, rutherfordium, dubnium, seaborgium, bohrium, hassium, meitnerium, darmstadtium, roentgenium, and the like. Examples of the counterions in the metal salts include, but are not limited to, nitrate, nitrite, phosphate, phosphite, sulfate, sulfite, fluoride, chloride, bromide, iodide, acetate, carbonate and the like. Any of the metals may be in any of the salt forms and selected based on the ability of the metal salt (metal/counterion combination) to dissolve in the solvent. Said salts may be hydrates, alcoholates, or acetonates.

Examples of organic linkers include, but are not limited to, 4,5-dicyanoimidazole, a substituted 4,5 dicyanoimidazole, oxalic acid, ethyloxalic acid, fumaric acid, 1,3,5-benzene tribenzoic acid (BTB), DCPB, benzene tribiphenylcarboxylic acid (BBC), 5,15-bis (4-carboxyphenyl) zinc (II) porphyrin (BCPP), 1,4-benzene dicarboxylic acid (BDC), 2-amino-1,4-benzene dicarboxylic acid (R3-BDC or H2N BDC), 1,1′-azo-diphenyl 4,4′-dicarboxylic acid, cyclobutyl-1,4-benzene dicarboxylic acid (R6-BDC), benzene tricarboxylic acid, 2,6-naphthalene dicarboxylic acid (NDC), 1,1′-biphenyl 4,4′-dicarboxylic acid (BPDC), 2,2′-bipyridyl-5,5′-dicarboxylic acid, adamantane tetracaboxylic acid (ATC), adamantane dibenzoic acid (ADB), dihydroxyterephthalic acid (DHBDC), biphenyltetracarboxylic acid (BPTC), tetrahydropyrene 2,7-dicarboxylic acid (HPDC), hihydroxyterephthalic acid (DHBC), pyrene 2,7-dicarboxylic acid (PDC), pyrazine dicarboxylic acid, acetylene dicarboxylic acid (ADC), camphor dicarboxylic acid, fumaric acid, benzene tetracarboxylic acid, 1,4-bis(4-carboxyphenyl)butadiyne, nicotinic acid, and terphenyl dicarboxylic acid (TPDC), 2,5-dihydroxy-1,4-benzene-dicarboxylic acid (H₄DOBDC), 4,4′-dihydroxybiphenyl-3,3′-dicarboxylic acid (H₄DOBPDC), 4,4″-dihydroxy-[1,1′:4′,1″-terphenyl]-3,3″-dicarboxylic acid (H₄DOTPDC), 3,3′-dihydroxybiphenyl-4,4′-dicarboxylic acid (pc-DOBPDC) and the like, substituted derivatives of the aforementioned linkers, and the like, and any combination of the totality thereof.

Examples of bases include, but are not limited to, piperazine, 1,4-dimethylpiperazine, sodium hydroxide, potassium hydroxide, lithium hydroxide, ammonium hydroxide and the like, and any combination thereof.

Examples of acids include, but are not limited to, hydrochloric acid, nitric acid, citric acid, oxalic acid, malonic acid, succinic acid, glutaric acid, acetic acid, perchloric acid, phosphoric acid, phosphorus acid, sulfuric acid, formic acid, hydrofluoric acid, and the like, and any combination thereof.

Examples of acids and conjugate bases, and bases and conjugate acids which are used to buffer the nominal pH include, but are not limited to, acetic acid/acetate, citric acid/citrate, boric acid/borate, and the like, the buffers known as “Good Buffers” defined in Biochemistry, 1966, 5, 467-477, and the noncomplexing tertiary amine buffers known as “Better Buffers” defined in Anal Chem., 1999, 71, 3140-3144.

### Example Embodiments

A first nonlimiting example embodiment of the present disclosure is a method comprising: identifying synthesis conditions that includes a first solvent system by which a metal-organic framework is successfully synthesized; and synthesizing the metal-organic framework under substantially the same synthesis conditions but with a second solvent system (or a comparable solvent system) having a difference (Rₐ) between Hansen solubility parameters of 5 MPa^{0.5} or less as compared to the first solvent system. Said method may further include one or more of the following: Element 1: where the second solvent system has a comparable dispersion parameter (δ_{D}) and a comparable polarity parameter (δ_{P}) and a comparable hydrogen bonding parameter (δ_{H}) relative to the first solvent system; Element 2: wherein the metal-organic framework is MOF-274; Element 3: Element 2 and wherein the synthesis conditions includes a linker comprising 4,4′-dihydroxybiphenyl-3,3′-dicarboxylic acid (H₄DOBPDC); Element 4: Element 2 and wherein the first solvent system and the second solvent system have a dispersion parameter (δ_{D}) of 15 MPa^{0.5} to 17 MPa^{0.5} and the polarity parameter (δ_{P}) of 6 MPa^{0.5} to 15 MPa^{0.5} and the hydrogen bonding parameter (δ_{H}) of 12 MPa^{0.5} to 18 MPa^{0.5}; Element 5: Element 2 and wherein the second solvent system comprises at least one solvent selected from the group consisting of tetrahydrofuran, water, acetonitrile, acetone, toluene, dimethylsulfoxide, methanol, n-propanol, and ethanol; Element 6: wherein the second solvent system is absent dimethylformamide; Element 7: Element 4 and wherein the second solvent system comprises is selected from the group consisting of: (a) 26 vol % water, 54 vol % acetone, and 20 vol % dimethylsulfoxide, (b) 28 vol % water and 72 vol % acetone, (c) 1 vol % to 65 vol % water, 5 vol % to 70 vol % acetonitrile, and 5 vol % to 50 vol % tetrahydrofuran, (d) 5 vol % to 40 vol % water, 5 vol % to 30 vol % acetonitrile, 1 vol % to 20 vol % tetrahydrofuran, 10 vol % to 75 vol % n-propanol, and 5 vol % to 25 vol % toluene, (e) 10 vol % to 20 vol % water, 5 vol % to 20 vol % acetonitrile, 5 vol % to 20 vol % tetrahydrofuran, 30 vol % to 50 vol % n-propanol, 1 vol % to 20 vol % toluene, and 1 vol % to 10 vol % ethanol, (f) 1 vol % to 15 vol % water, 65 vol % to 90 vol % n-propanol, 1 vol % to 20 vol % toluene, and 1 vol % to 20 vol % ethanol, and (g) 1 vol % to 15 vol % water, 70 vol % to 98 vol % n-propanol, and 1 vol % to 15 vol % toluene, wherein each of (a)-(g) have a total vol % is 100; and Element 8: wherein the synthesis conditions comprise a synthesis solution being buffered to maintain a nominal pH. Examples of combination of elements include, but are not limited to, Element 2 in combination with two or more of Elements 3-7; and Elements 1 and 2 in combination optionally in further combination with one or more of Elements 3-7; two or all of Elements 1, 2, and 8 in combination; and Elements 2 and 8 in combination with one or more of Elements 3-7.

Another nonlimiting example embodiment is a method comprising: performing a plurality of metal-organic framework syntheses under two or more of substantially the same synthesis conditions that vary a solvent composition based on a Hansen solubility parameter; and identifying a range for the Hansen solubility parameter under which the metal-organic framework can be synthesized. Said method may further include one or more of the following: Element 8: the method further comprising: synthesizing additional metal-organic frameworks under substantially the same synthesis conditions that vary the solvent composition within the range for the Hansen solubility parameter; Element 9: wherein varying the solvent composition includes using solvents that maintain a comparable dispersion parameter (δ_{D}) and a comparable polarity parameter (δ_{P}) while varying a hydrogen bonding parameter (δ_{H}); Element 10: wherein varying the solvent composition includes using solvents that maintain a comparable δ_{H} and a comparable δ_{H} while varying a δ_{P}; and Element 11: wherein varying the solvent composition includes using solvents that maintain a comparable δ_{P} and a comparable δ_{H} while varying a δ_{D}. In this example, within the plurality of metal-organic framework syntheses two or more Elements 9-11 can be satisfied. Element 9 can be in combination with one or more of Elements 9-11.

Yet another nonlimiting example embodiment is a method comprising: performing a plurality of metal-organic framework syntheses under two or more of substantially the same synthesis conditions that vary a solvent composition based on a Hansen solubility parameter; characterizing a morphology characteristic of the metal-organic frameworks; identifying trends in the morphology characteristic as a function of the Hansen solubility parameter; and synthesizing a metal-organic framework under substantially the same synthesis conditions with a solvent having the Hansen solubility parameter chosen to achieve a desired morphology characteristic based on the identified trends. Said method may further include one or more of the following: Element 12: wherein characterizing the morphology includes one or more of the following characteristics: aspect ratio, surface area, pore size, composition, and crystal structure; Element 13: wherein varying the solvent composition includes using solvents that maintain a comparable dispersion parameter (δ_{D}) and a comparable polarity parameter (δ_{P}) while varying a hydrogen bonding parameter (δH); Element 14: wherein varying the solvent composition includes using solvents that maintain a comparable δ_{D} and a comparable δ_{H} while varying a δ_{P}; and Element 15: wherein varying the solvent composition includes using solvents that maintain a comparable δ_{P} and a comparable δ_{H} while varying a δ_{H} In this example, within the plurality of metal-organic framework syntheses two or more Elements 13-15 can be satisfied. Element 12 can be in combination with one or more of Elements 13-15.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### Examples

Example 1. MOF-274 synthesis conditions (see representative MOF-274 synthesis conditions below) were optimized and determined that a solvent mixture of 55% methanol and 45% N,N-dimethylformamide (DMF) provided desired needle morphologies, powder x-ray diffraction patterns, and surface areas. The synthesis conditions were then repeated with three different solvents having comparable Hansen solubility parameters to the solvent mixture of 55% methanol and 45% N,N-dimethylformamide, with nominal pH fixed through the addition of MOPS and/or sodium MOPS, see Table 1.

TABLE 1
Solvent systems, provided in percent volume, with corresponding
Hansen solubility parameters and the Rₐ value relative
to the 55% methanol, 45% DMF solvent system.

| Solvent | δ_{D} | δ_{P} | δ_{H} | Rₐ |
|---|---|---|---|---|
| 55% methanol | 16.14 | 12.93 | 17.35 | N/A |
| 45% DMF | | | | |
| 26% water | 16.08 | 13.06 | 16.82 | 0.56 |
| 54% acetone | | | | |
| 20% DMSO | | | | |
| 28% water | 15.5 | 12.0 | 16.88 | 1.67 |
| 72% acetone | | | | |
| 25% water | 15.92 | 12.83 | 15.87 | 1.54 |
| 37% acetonitrile | | | | |
| 38% THF | | | | |

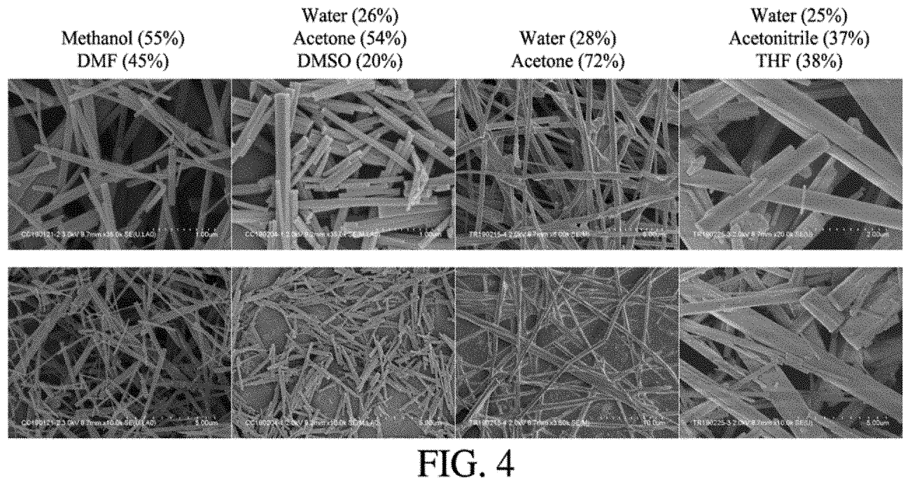

FIG. 4 includes scanning electron micrographs of the MOFs synthesized under the same synthesis conditions except with different solvents having comparable Hansen solubility parameters. All four solvents successfully produced MOFs with needle morphologies and comparable aspect ratios.

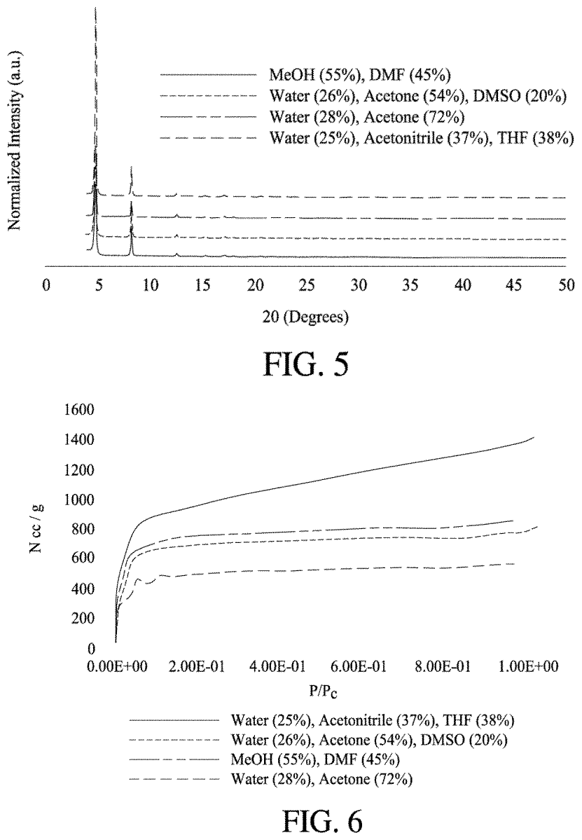

FIG. 5 is a plot of the powder x-ray diffraction data for the MOFs synthesized under the same synthesis conditions except with different solvents having comparable Hansen solubility parameters. This illustrates that the crystal structure of the produced MOFs are comparable.

FIG. 6 is a plot of the surface area for the MOFs synthesized under the same synthesis conditions except with different solvents having comparable Hansen solubility parameters. This illustrates that the surface area of the produced MOFs are very close.

Example 2. Another approach to MOF synthesis is to use different solvents based on varied Hansen solubility parameters to determine a range for the Hansen solubility parameters under which the MOF can be synthesized. That is, a first screening of solvents can be based on significantly varied individual Hansen solubility parameters.

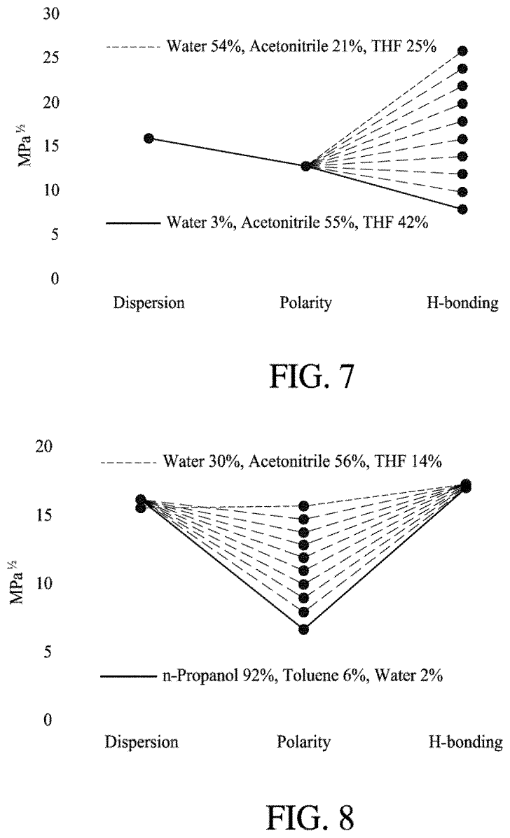

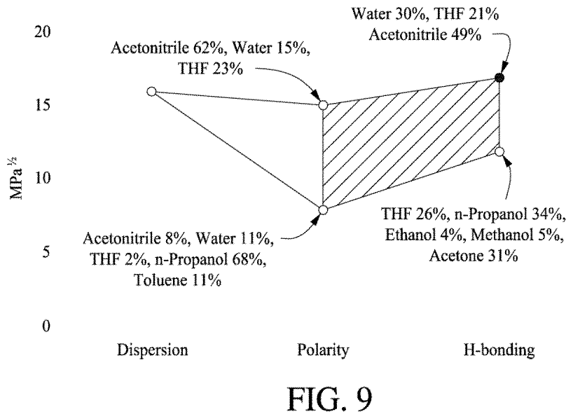

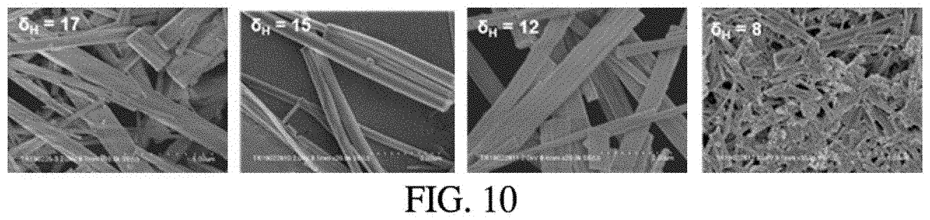

For example, the relative concentrations of water, acetonitrile, and THF in a solvent mixture can be used to maintain a comparable δ_{D} and a comparable δ_{P} while greatly varying the δ_{H}, for example from 8.0 MPa^{0.5} to 26.0 MPa^{0.5} as illustrated in FIG. 7. Further, the relative concentration and composition of solvent mixtures can be used to maintain a comparable δ_{H} and a comparable δ_{H} while greatly varying the δ_{P}, for example from 6.7 MPa^{0.5} to 15.7 MPa^{0.5} as illustrated in FIG. 8. In FIGS. 7 and 8, the solid lines represent the indicated solvents, and the dashed lines represent potential intermediate values that could be explored. Provided that a MOF can be synthesized in each of these boundary solvent conditions, the combination of two graphs, as illustrated in FIG. 9, provides the range for the Hansen solubility parameters under which the MOF can be synthesized. Knowing this range of Hansen solubility parameters can provide researchers with guidance for other solvents to try in further experimentation.

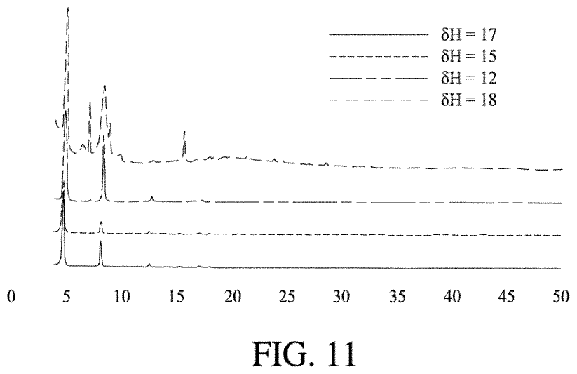

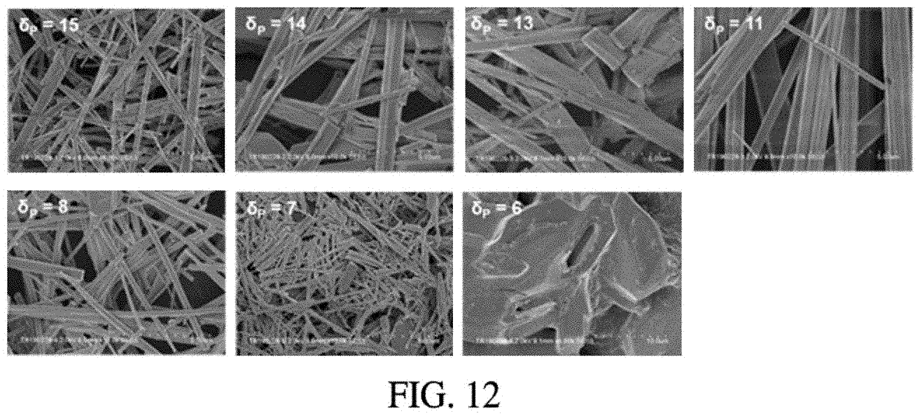

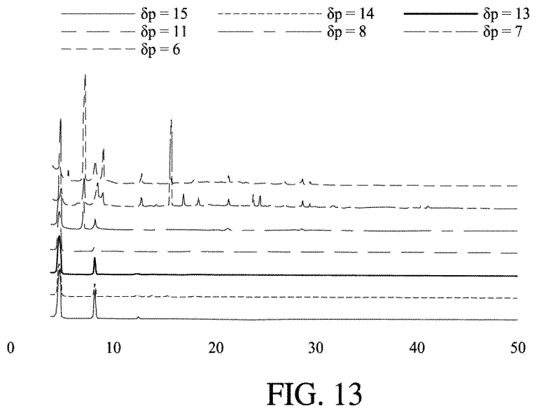

In the present example, these boundary solvent conditions and some intermediate solvent conditions (see Table 2) were used to synthesize MOF-274. FIGS. 10 and 11 are scanning electron micrographs and a plot of the powder x-ray diffraction data, respectively, for MOFs synthesized with variable δ_{H}. FIGS. 12 and 13 are scanning electron micrographs and a plot of the powder x-ray diffraction data, respectively, for MOFs synthesized with variable δ_{P}. This example illustrates that the range for the Hansen solubility parameters under which the MOF can be synthesized can be determined and is useful in exploring solvent effects without undo experimentation of overlapping solvent systems where all three Hansen solubility parameters are comparable.

TABLE 2

| | | | |
|---|---|---|---|
| δ_{D} | δ_{P} | δ_{H} | Solvent System (vol %) |

Variable δ_{H}

| | | | |
|---|---|---|---|
| 15.91 | 12.82 | 26.00 | water 53%, acetonitrile 13%, THF 33% |
| 15.91 | 12.82 | 24.00 | water 48%, acetonitrile 18%, THF 34% |
| 15.91 | 12.82 | 22.00 | water 42%, acetonitrile 23%, THF 35% |
| 15.92 | 12.82 | 20.00 | water 36%, acetonitrile 27%, THF 36% |
| 15.92 | 12.82 | 18.00 | water 31%, acetonitrile 32%, THF 37% |
| 15.92 | 12.82 | 17.00 | water 28%, acetonitrile 34%, THF 38% |
| 15.92 | 12.82 | 16.00 | water 25%, acetonitrile 37%, THF 38% |
| 15.92 | 12.83 | 15.00 | water 23%, acetonitrile 39%, THF 38% |
| 15.92 | 12.83 | 14.00 | water 20%, acetonitrile 41%, THF 39% |
| 15.92 | 12.83 | 13.00 | water 17%, acetonitrile 44%, THF 39% |
| 15.92 | 12.83 | 12.00 | water 14%, acetonitrile 46%, THF 40% |
| 15.93 | 12.83 | 11.00 | water 11%, acetonitrile 48%, THF 40% |
| 15.93 | 12.83 | 10.00 | water 9%, acetonitrile 51%, THF 40% |
| 15.93 | 12.83 | 8.00 | water 3%, acetonitrile 55%, THF 42% |

Variable δ_{P}

| | | | |
|---|---|---|---|
| 15.45 | 16.66 | 17.35 | acetonitrile, 63%, water 31%, THF 6% |
| 15.56 | 15.72 | 17.35 | acetonitrile 56%, water 30%, THF 14% |
| 15.68 | 14.78 | 17.35 | acetonitrile 49%, water 30%, THF 21% |
| 15.80 | 13.83 | 17.35 | water 30%, THF 29%, acetonitrile 41% |
| 15.91 | 12.89 | 17.35 | acetonitrile 34%, water 29%, THF 37% |
| 16.14 | 12.00 | 17.35 | acetonitrile 31%, water 32%, THF 13%, acetone 1%, toluene 21% |
| 16.14 | 11.00 | 17.35 | acetonitrile 25%, water 27%, THF 12%, n-propanol 18%, toluene 18% |
| 16.14 | 10.00 | 17.35 | acetonitrile 19%, water 21%, THF 12%, n-propanol 34%, toluene 14% |
| 16.13 | 9.00 | 17.35 | acetonitrile 12%, water 16%, THF 11%, n-propanol 44%, ethanol 6%, toluene 11% |
| 16.13 | 8.00 | 17.35 | acetonitrile 8%, water 11%, THF 2%, n-propanol 68%, toluene 11% |
| 16.14 | 7.00 | 17.35 | water 5%, toluene 9%, n-propanol 75%, ethanol 11% |
| 16.10 | 6.70 | 17.09 | n-propanol 92%, toluene 6%, water 2% |

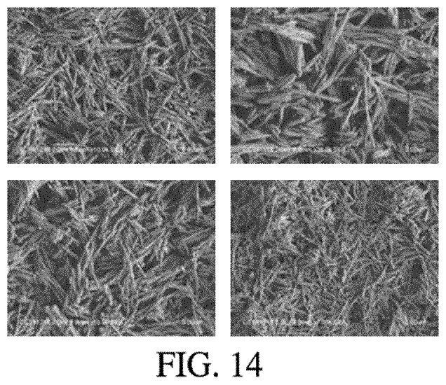

Example 3. This example illustrates that MOF morphology can be controlled or otherwise tuned through the application and understanding of Hansen solubility parameters. The MOF-274 was synthesized in the traditional solvent of 10% water, 25% toluene, and 65% DMSO to produce MOF structures with a short, needle-like morphology, see FIG. 14. In contrast, changing the solvent as done in Examples 2 and 3 produced MOFs with a longer, rod-like morphology.

Representative Synthesis Conditions of MOF-274: Syntheses scale linearly to larger/smaller volumes. The described synthesis is for the 125-mL scale.

All solvents were purged with N₂ to remove oxygen. To 75 mL of solvent, 431.6 mg (1.68 mmol) Mg(NO₃)₂.6H₂O, 37.6 mg (0.19 mmol) MnCl₂.4H₂O, and 205.48 (0.75 mmol) H₄DOBPDC (4,4′-dihydroxy-(1,1′-biphenyl)-3,3′-dicarboxylic acid) linker were added and stirred until full dissolution. 392.5 mg (1.875 mmol) 3-(N-morpholino)propanesulfonic acid (MOPS) and 1.3 g (5.625 mmol) 3-(N-morpholino)propanesulfonate (NaMOPS) is added to the solution and stirred to complete dissolution. In this instance, a buffer was not used. The reaction solution is transferred to a 125-mL polytetrafluoroethene-lined autoclave. The autoclave is sealed and heated at 120° C. for 2 days, and then cooled naturally to room temperature. The solids are collected by centrifugation and the resulting material is washed with DMF and methanol multiple times. The final material is stored in methanol.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
identifying synthesis conditions that include a first solvent system by which a metal-organic framework is successfully synthesized; and
synthesizing the metal-organic framework under substantially the same synthesis conditions but with a second solvent system that is different from the first solvent system, wherein the second solvent system has a difference (Rₐ) between Hansen solubility parameters of 5 MPa^{0.5} or less as compared to the first solvent system.

2. The method of claim 1, where the second solvent system has a comparable dispersion parameter (δ_{D}) and a comparable polarity parameter (δ_{P}) and a comparable hydrogen bonding parameter (δ_{H}) relative to the first solvent system.

3. The method of claim 1, wherein the metal-organic framework is MOF-274.

4. The method of claim 3, wherein the synthesis conditions includes a linker comprising 4,4′-dihydroxybiphenyl-3,3′-dicarboxylic acid (H₄DOBPDC).

5. The method of claim 3, wherein the second solvent system and the first solvent system have a dispersion parameter (δ_{D}) of 15 MPa^{0.5} to 17 MPa^{0.5} and the polarity parameter (δ_{P}) of 6 MPa^{0.5} to 15 MPa^{0.5} and the hydrogen bonding parameter (δ_{H}) of 12 MPa^{0.5} to 18 MPa^{0.5}.

6. The method of claim 3, wherein the second solvent system comprises at least one solvent selected from the group consisting of tetrahydrofuran, water, acetonitrile, acetone, toluene, dimethylsulfoxide, methanol, n-propanol, and ethanol.

7. The method of claim 6, wherein the second solvent system is absent dimethylformamide.

8. The method of claim 7, wherein the second solvent system comprises is selected from the group consisting of: (a) 26 vol % water, 54 vol % acetone, and 20 vol % dimethylsulfoxide, (b) 28 vol % water and 72 vol % acetone, (c) 1 vol % to 65 vol % water, 5 vol % to 70 vol % acetonitrile, and 5 vol % to 50 vol % tetrahydrofuran, (d) 5 vol % to 40 vol % water, 5 vol % to 30 vol % acetonitrile, 1 vol % to 20 vol % tetrahydrofuran, 10 vol % to 75 vol % n-propanol, and 5 vol % to 25 vol % toluene, (e) 10 vol % to 20 vol % water, 5 vol % to 20 vol % acetonitrile, 5 vol % to 20 vol % tetrahydrofuran, 30 vol % to 50 vol % n-propanol, 1 vol % to 20 vol % toluene, and 1 vol % to 10 vol % ethanol, (f) 1 vol % to 15 vol % water, 65 vol % to 90 vol % n-propanol, 1 vol % to 20 vol % toluene, and 1 vol % to 20 vol % ethanol, and (g) 1 vol % to 15 vol % water, 70 vol % to 98 vol % n-propanol, and 1 vol % to 15 vol % toluene, wherein each of (a)-(g) have a total vol % is 100.

9. The method of claim 1, wherein the synthesis conditions comprise a synthesis solution being buffered to maintain a nominal pH.

10. A method comprising:
performing a plurality of metal-organic framework syntheses under two or more of substantially the same synthesis conditions that vary a solvent composition based on a Hansen solubility parameter; and
identifying a range for the Hansen solubility parameter under which the metal-organic framework can be synthesized.

11. The method claim 10 further comprising:
synthesizing additional metal-organic frameworks under substantially the same synthesis conditions that vary the solvent composition within the range for the Hansen solubility parameter.

12. The method claim 10, wherein varying the solvent composition includes using solvents that maintain a comparable dispersion parameter (δ_{D}) and a comparable polarity parameter (δ_{P}) while varying a hydrogen bonding parameter (δ_{H}).

13. The method claim 10, wherein varying the solvent composition includes using solvents that maintain a comparable δ_{H} and a comparable δ_{H} while varying a δ_{P}.

14. The method claim 10, wherein varying the solvent composition includes using solvents that maintain a comparable δ_{P} and a comparable δ_{H} while varying a δ_{D}.

15. A method comprising:
performing a plurality of metal-organic framework syntheses under two or more of substantially the same synthesis conditions that vary a solvent composition based on a Hansen solubility parameter;
characterizing a morphology characteristic of the metal-organic frameworks;
identifying trends in the morphology characteristic as a function of the Hansen solubility parameter; and
synthesizing a metal-organic framework under substantially the same synthesis conditions with a solvent having the Hansen solubility parameter chosen to achieve a desired morphology characteristic based on the identified trends.

16. The method of claim 15, wherein characterizing the morphology includes one or more of the following characteristics: aspect ratio, surface area, pore size, composition, and crystal structure.

17. The method of claim 15, wherein varying the solvent composition includes using solvents that maintain a comparable dispersion parameter (δ_{D}) and a comparable polarity parameter (δ_{P}) while varying a hydrogen bonding parameter (δ_{H}).

18. The method of claim 15, wherein varying the solvent composition includes using solvents that maintain a comparable δ_{H} and a comparable δ_{H} while varying a δ_{P}.

19. The method of claim 15, wherein varying the solvent composition includes using solvents that maintain a comparable δ_{P} and a comparable δ_{H} while varying a δ_{D}.
